(19) United States
(12) Patent Application Publication
(10) Pub. No.: US 2024/0398482 A1
(43) Pub. Date: Dec. 5, 2024

(54) Method of Simulating the Fitting of Implantable Medical Devices Inside a Patient's Anatomy

(71) Applicant: OXFORD HEARTBEAT LTD., London, England (GB)

(72) Inventors: Francesco Iori, London (GB); Katerina Spranger, London (GB)

(21) Appl. No.: 18/725,545

(22) PCT Filed: Dec. 23, 2022
(86) PCT No.: PCT/EP2022/087773

§ 371 Date: Jun. 28, 2024

(30) Foreign Application Priority Data
Dec. 31, 2021 (EP) 21218506.0

(51) Int. Cl. A61B 34/10; A61B 17/12; A61B 34/00

(52) U.S. Cl. CPC A61B 34/10; A61B 17/12109; A61B 34/25; A61B 2034/104; A61B 2034/105; A61B 2034/108

(57) ABSTRACT

Broadly speaking, the present invention provides a technical solution by which the performance of one or more implantable medical devices is simulated to determine a best fit device for a patient's vessel. This technical solution advantageously ensures that when planning the deployment of an implantable medical device, a clinician is able to take into account many of the important factors for deployment, such as wall apposition, porosity, reduction and obstructions of flow, and vessel geometry. Additionally, providing a computationally efficient method by which to carry out this planning of the deployment of an implantable medical device.

### FIELD OF INVENTION

This invention relates generally to the simulation of implantable medical devices, such as flow-diverting or implantable intravascular devices, inside a patient's anatomy in order to determine a best fit device prior to a surgical procedure.

### BACKGROUND

Clinicians who use implantable medical devices, such as stents or other intravascular or implantable neurovascular devices, often deploy them in a radially compressed state in a delivery system to parts of the patient's body that are difficult to access. The implantable medical devices are then deployed from the delivery system once they are positioned. The effective functioning of a device after a particular procedure is dependent on the specific final deployed dimensions and positioning of the implantable medical device. The quality of the “fit” of the device when in this final orientation is dependent on many factors, including the properties of the implantable device, the forces applied during deployment and the forces applied by the patient's anatomy.

If an implantable medical device is too short after deployment, it may not sufficiently fulfill its intended purpose. Conversely, if an implantable medical device is too long after deployment, it can obstruct flows which are not intended to be obstructed by the implantable medical device.

The implantable medical device may have a variable diameter in its deployed configuration along its longitudinal axis and may extend or contract in length along its longitudinal axis in relation to its diameter along its longitudinal axis. Therefore, if the diameter of the implantable medical device is incorrect, it may subsequently impact the length of the device, causing the above discussed issues. A device that does not fit correctly may further result in unsatisfactory performance, migration of the device from its intended position and/or cause thrombo-embolic complications. In addition to the overall deployed dimensions of the device, the features of the specific final configuration, such as the arrangement of wires, also impacts on the performance of an implantable device.

It is therefore important for the clinician, or other medical professional, to ensure a correctly sized implantable medical device, with appropriate properties for a particular application, is used and that they plan where they will deploy the implantable medical device from the delivery system. Particularly, as the radially compressed implantable medical device will expand into the area it has been deployed and its final configuration will be dependent on a number of factors, meaning it is difficult to predict with precision the final configuration of the device after deployment.

Current systems to enable a clinician to plan the deployment of an implantable medical device often utilize two-dimensional or three-dimensional images of the deployment location in the patient. However, utilizing these systems, the clinician is not able to take into account many of the important factors discussed above to determine a best fit device for a particular procedure in an objective and reproducible way. Therefore decisions made by individual clinicians may vary greatly due to personal preference and experience.

There is hence a need for software to assist the clinician in determining an implantable medical device with the properties to provide the best performance when deployed in a particular area within a patient's body and in determining where best to position the selected implantable medical device, before commencing a surgical procedure to deploy the device in a patient.

### SUMMARY OF THE INVENTION

In a first aspect, the invention provides a computer-implemented method for simulating the deployment of a plurality of implantable medical devices to determine a best fit implantable medical device for a target deployment location within a patient vascular structure, the method comprising: receiving image data corresponding to the patient's vascular structure; creating a three-dimensional model of the patient's vascular structure based on the image data; simulating the deployed configuration of the plurality of implantable medical devices at a target location within the three-dimensional model of the vascular structure; determining a suitability metric for each simulated implantable medical device, the suitability metric providing a measure of the fit of the deployed implantable medical device within the patient's vascular structure; and outputting an indication of a best fit implantable medical device based on the suitability metric.

By simulating the deployed orientation of a plurality of implantable medical devices and calculating a suitability metric for each of the simulated devices, a clinician, or a medical professional, is able to determine a best fit device prior to surgery. Given that it can be difficult to accurately predict the influence of the properties of the device, the patient's anatomy and the deployment procedure on the performance of the device prior to surgery, the present method allows for an accurate prediction of a best fit device to help guide a clinician in selecting the best fitting device for a particular procedure. By determining a best fit device prior to surgery, the likelihood of success is greatly increased, meaning better patient outcomes and a reduction in the likelihood of subsequent surgeries. By automating the selection of a best fit device, a more objective measure is provided in comparison to existing methods which rely significantly on clinician's personal preferences and biases based on prior experience.

Broadly speaking, the present invention provides a technical solution by which intravascular implantable medical devices can be simulated inside a patient's anatomy in order to determine a best fit device. This technical solution advantageously ensures that when planning the deployment of an implantable medical device, a clinician, or other medical professional, is able to take into account many of the important factors for deployment, such as wall apposition, porosity, reductions and obstructions of flow, and vessel geometry in a readily understandable and automatically generated metric. Additionally, the present invention provides a computationally efficient method by which to carry out this planning of the deployment of an implantable medical device.

As used herein, the term implantable medical device is intended to mean a medical device for implanting in a bodily cavity, particularly a vascular structure, for example through a minimally invasive surgical procedure.

In a preferable embodiment, the implantable medical devices are implantable intravascular devices, flow-diverting devices such as braided stents or intrasaccular devices, but they may alternatively be stent grafts, laser-cut stents, or implantable neurovascular medical devices. Implantable neurovascular medical devices may include, for example, stents such as arterial stents, intracranial stents, or carotid stents, embolic coils, aneurysm coils, flow diverters, embolic protection devices, or neurothrombectomy devices. Flow-diverting devices are devices used to treat pathological blood vessels which present an aneurysm, by occluding it and redirecting the normal blood flow. Stent grafts are often used for the treatment of aortic aneurysms and dissections, and laser-cut stents also for the treatment of stenosis. Embolic coils are often used to treat intracranial aneurysms by inducing clotting

Devices or tools used when implanting implantable medical devices, such as catheters, balloon systems, guidewires, or stent retrievers, may be additionally simulated when simulating the deployed configuration of a plurality of implantable medical devices at a target location. Said devices or tools may be simulated to simulate their use when implanting an implantable medical device or their use when withdrawing from implanting an implantable medical device. Alternatively or additionally, said devices or tools may be simulated to assist in the simulation of the deployment of implantable medical devices.

The target location is a location within the vascular structure at which the implantable medical device is to be implanted. The target location may be defined between a start point and an end point, where preferably the implantable medical device is deployed at the starting point and extends towards the end point during deployment. The target location is preferably a portion of vessel within the vascular structure, i.e. a length of vessel between a distal point and a proximal point. The implantable device is preferably deployed starting at the distal point and extends towards the proximal point during deployment. The length between the start (distal) and end (proximal) point of the target location is defined as the target length. The proportion of the target length over which the device extends (from the distal point towards the proximal point) will depend on a number of factors, including one or more of the unconstrained length of the implantable device (the length of the device in a fully expanded state assuming no external forces are applied), the forces applied by the patient's vascular structure, the forces applied by the delivery system during deployment and the mechanical properties of the device. In some examples the target location may be within an aneurysm. This is particularly the case for intrasaccular devices. In this case the target location may be defined by the internal walls of the aneurysm.

Preferably simulating the deployed configuration of the plurality of implantable medical devices at a target location within the three-dimensional model of the vascular structure comprises performing a numerical simulation of the expansion of the implantable medical device based on one or more properties of the implantable medical device and one or more properties of the patient's vascular structure. In particular the simulation may comprise a mechanical and/or geometrical simulation of the expansion of the device. More specifically, preferably the numerical simulation comprises simulating the expansion of the implantable medical device within the constraints imposed by the geometry of the patient's vascular structure. In some examples, the simulation comprises simulating the forces applied to the implantable medical device by the patient's vascular structure and, optionally, a delivery system used to deploy the device. The method may comprise numerically simulating the expanded configuration of the implantable medical device within the constraints imposed by the patient's vascular geometry as defined in the three-dimensional model, where the numerical simulation is preferably also based on mechanical constraints of the implantable medical device, for example the braiding of the wires of the device.

In some examples simulating the deployed configuration of the implantable medical device may comprise the following steps:
a. determine a ratio indicating the change in length of the stent as a function of the local morphology of the vascular structure
b. obtain the three-dimensional centreline from the three dimensional model of the vascular structure;
c. define the location of the starting point at which the device will be placed in the three-dimensional model of the vascular structure;
d. divide the centreline of the vascular structure into a plurality of segments;
e. determine the descriptive parameters of the morphology of said vascular structure for the first of the plurality of segments, where the descriptive parameters comprise geometrical parameters and/or mechanical factors;
f. calculate the length of the stent for said first segment using the indicator ratio of step a)
g. subtract said length of the segment calculated in step f) from the nominal length of the stent in order to obtain a new nominal length; if said new nominal length is different from 0 then steps e) to g) will be repeated for the segment contiguous with the preceding segment; if the new nominal length is approximately 0, all the distances of each segment will be added together, and this sum will be the final length of said stent after its positioning.

The ratio of step a) may be determined based on mathematical modeling.

The suitability metric preferably comprises a measure of the correspondence between a dimension of the implantable medical device in the deployed configuration and a corresponding dimension of the target location. In this way, the assessment of best fit is based on the correspondence of the dimensions of the device within the deployed configuration relative to the surrounding target location. The dimensions of the device within the deployed configuration may comprise length, volume, thickness, diameter, or height. Preferably the target location is a vessel portion having a target length between a distal and proximal position and the suitability metric provides a measure of the correspondence between the deployed length of the device and the target length of the target location. Alternatively, the target location may be an aneurysm having a target diameter and the suitability metric provides a measure of the correspondence of the deployed diameter of the device and the target diameter. The measure of the correspondence between a dimension of the device in the deployed configuration and a corresponding dimension of the device may be referred to as a dimension index. The dimension index may be in the form of a value between 0 and 1, where 1 indicates a best fit and 0 indicates a poor fit. The dimension index may take a value of 1 where the dimension of the deployed device is within a predetermined range of the target dimension.

The suitability metric preferably comprises a measure of the apposition between the implantable medical device in the deployed configuration and a wall of the vascular structure, in particular a measure of the apposition between the surface of the implantable device in the deployed configuration and the adjacent wall of a vessel, where a portion of the vessel wall corresponding to an aneurysm neck is preferably excluded. The measure of apposition may be referred to as an apposition index. The measure of the apposition preferably comprises a measure of the distance between an outer surface of the implantable medical device in the deployed configuration and the wall of the vascular structure. Preferably the method may comprise determining an average distance between the outer surface of the implantable medical device in the deployed configuration on the wall of the vascular structure, preferably excluding a portion of the surface area of the implantable medical device corresponding to an aneurysm neck.

The measure of the apposition preferably comprises an apposition index comprising a percentage of the surface of the implantable medical device that is within a threshold distance from the wall of the vascular structure, preferably excluding a portion of the surface area of the implantable medical device corresponding to an aneurysm neck. The threshold distance may be between 0.1 and 1 mm, preferably 0.5 mm, where a separation between the surface of the implantable medical device and the adjacent wall of the vascular structure within the range indicates good apposition (and is therefore indicative of a good fitting device).

Where the target location comprises a vessel comprising an aneurysm, the apposition index may be calculated by determining a portion of the vessel corresponding to the aneurysm neck; excluding the portion of the outer surface of the implantable medical device corresponding to the determined portion of the vessel; calculating the apposition index over the remainder of the outer surface of the implantable medical device. In this way, an improved measure of the apposition is provided given the apposition around the aneurysm is not expected. Determining the portion of a vessel or vascular structure corresponding to the aneurysm neck may comprise one or more of: determining a portion of the vessel where the radius of the vessel portion is greater than the average radius over the remainder of the vessel in the target location; determining a portion of the vessel where the radius of the vessel portion is greater than a centerline vessel radius by more than a threshold amount; determining a portion of the vessel where there is a peak in the vessel radius along the length of the vessel in the target location.

The centerline radius may be defined as the minimum distance between the centerline of a vessel and the vessel wall or the average distance between the centerline of the vessel and the vessel wall.

Where the target location comprises a vessel comprising an aneurysm, the suitability metric may comprise a landing zone index, the landing zone index comprising: a measure of the length of the landing zones of the implantable medical device, where the landing zones comprise longitudinal sections of the deployed implantable medical device positioned distally and proximally to an aneurysm neck in the target location, and where the landing zone index indicates a better fitting device where the landing zone length is above a threshold value or within a threshold range. When the landing zone length is above the threshold value, this ensures good purchase, good apposition and avoids device migration.

By including a measure of the length of the landing zones of the implantable intravascular device or flow-diverting device in the suitability metric, the best fit device for a target deployment location within a patient vascular structure can be more accurately determined, particularly because the landing zone indicates a better fitting device where the landing zone length is above a threshold value and preferably where the apposition within the landing zones is above a threshold value. The landing zone index in some embodiments may provide a measure of the length of the longitudinal sections of the device positioned proximally and distally to an aneurysm neck that have an apposition index above a threshold value.

The landing zone index may comprise a measure of the fitting of the landing zones of the device. The landing zone index may comprise a measure of the percentage difference between the vessel diameter and the device diameter within the landing zones. The percentage difference may be determined over a length of the device in the landing zones corresponding to an optimal landing zone length. In one example the landing zone index may be calculated by integrating a weight function, which assigns a weight from 0 to 1 according to the fitting of the device, over the landing zones

The landing zone index may be calculated according to:

$$S = \frac{{S}_{d} + {S}_{p}}{2}$$

$$where ,$$

$${S}_{d} = \frac{1}{{L}_{opt}} {\int }_{0}^{{L}_{opt}} Wf ( SizingRatio ) ds$$

$${S}_{p} = \frac{1}{{L}_{opt}} {\int }_{{L}_{device} - {L}_{opt}}^{{L}_{device}} Wf ( SizingRatio ) ds$$

Where: Sₚ is the proximal landing zone sizing index; S_{d} is the distal landing zone sizing index; Lₒₚₜ is the optimal landing zone length for the implantable medical device; L_{device} is the length of the deployed implantable medical device; and Wƒ is a weight function.

The weight function, Wƒ, may take the value 1 where the device is optimally sized and less than 1 where it is suboptimally sized. For example, the weight function may assign a weight of 0 where the implantable medical device has a deployed diameter that is more than 1, 5 or 10% undersized relative to the diameter of the vessel in the landing zone, or more than 20, 40 or 60% oversized. The weight function may assign a weight of 1 where the implantable medical device has a deployed diameter that is between 0% and 20% oversized based on the size of the vessel in the landing zone.

The weight function may be a linearly increasing function in regions between the over/undersized range and the optimal size range, for example between 5% and 0% undersized (i.e., where the device has a diameter which is smaller than the vessel diameter by between 0% and 5% of the vessel diameter), taking a value of 0 at 5% undersized and 1 where the diameters are perfectly matched. Similarly, the weight function may be a linearly decreasing function between 20% and 60% oversized (i.e., where the deployed device has a diameter which is greater than the vessel diameter by between 20% and 60% of the vessel diameter), taking a value of 1 at 20% oversized and 0 at 60% oversized.

Preferably, the suitability metric comprises a porosity index. Preferably, the porosity index comprises a measure of the porosity of the flow diverting device in the deployed configuration.

By including a measure of the porosity of the implantable intravascular device or flow-diverting device in the deployed configuration in the suitability metric, the best fit device for a target deployment location within a patient vascular structure can be more accurately determined. Particularly because the porosity of an implantable intravascular device or flow-diverting device may change as a mesh of the device changes shape and expands and contracts in different areas, depending on its deployed position and configuration and this porosity can have an impact on the performance of the deployed device.

Where the target location comprises a vessel comprising an aneurysm, the porosity index may be calculated by: determining a portion of the vascular structure corresponding to an aneurysm neck; selecting the portion of the outer surface of the deployed implantable medical device corresponding to the determined portion of the vessel; determining the porosity of the selected portion of the outer surface of the deployed implantable medical device.

Determining the portion of a vessel or vascular structure corresponding to the aneurysm neck may comprise one or more of: determining a portion of the vessel where the radius of the vessel portion is greater than the average radius over the remainder of the vessel in the target location; determining a portion of the vessel where the radius of the vessel portion is greater than a centerline vessel radius by more than a threshold amount; determining a portion of the vessel where there is a peak in the vessel radius along the length of the vessel in the target location. The porosity index preferably is determined such that the porosity index indicates a better fitting device where the porosity is below a threshold value over the selected portion of the outer surface. The average porosity may be determined over the selected portion of the outer surface and compared to a threshold value. Alternatively a weight function may be integrated over the surface area of the selected portion, where the weight function takes a value of 1 where the porosity is within a target porosity range and a value of less than 1 where the porosity is outside of the porosity range. The weight function may be a linear function between 0 and 1 in a transition porosity range between a poor porosity range and an optimal porosity range.

Optionally, the suitability metric is based on an obstruction value providing a measure of the degree to which the deployed implantable intravascular device or flow-diverting device obstructs side branches within the three-dimensional model of the vascular structure, the obstruction value indicating a better fitting device where fewer side branches are obstructed.

By including an obstruction value providing a measure of the degree to which the deployed implantable intravascular device or flow-diverting device obstructs side branches within the three-dimensional model of the vascular structure, the best fit device for a target location within a patient vascular structure can be more accurately determined. Particularly because a device has better fit where fewer side branches are obstructed and flow disturbances are limited.

Optionally, the suitability metric is based on a vessel geometry value providing a measure of the degree to which the deployed implantable intravascular device or flow-diverting device extends across one or more bends in the three-dimensional model of the vascular structure that comprise a radius of curvature below a threshold, the vessel geometry value indicating a better fitting device where fewer bends are present in the deployed location of the device that are below the radius of curvature threshold.

By including a measure of the degree to which the deployed implantable intravascular device or flow-diverting device extends across one or more bends in the three-dimensional model of the vascular structure that comprise a radius of curvature below a threshold in the suitability metric, the best fit device for a target deployment location within a patient vascular structure can be more accurately determined. Particularly because deploying implantable intravascular devices or flow-diverting devices inside vessel bends is challenging and can result in poor apposition, as full expansion is more difficult when the implantable intravascular device or flow-diverting device is bent. Hence, a device has a better fit where fewer bends are below the radius of curvature threshold.

Preferably, the suitability metric comprises a flow diversion index, where the flow diversion index comprises a measure of the change in blood flow through the patient's vascular structure. Preferably the flow diversion index comprises a measure of the reduction of blood flow to an aneurysm, for example through the aneurysm neck. Preferably the flow diversion index is calculated by: simulating blood flow through the patient's vascular structure without the presence of an implantable medical device; simulating blood flow through the patient's vascular structure including the deployed configuration of the implantable medical devices; determining a flow diversion index comprising a measure of the change in blood flow due to the deployed implantable medical device. Preferably the target location comprises a vessel comprising an aneurysm and the measure in the change of blood flow due to the deployed implantable medical device comprises a measure of a reduction in blood flow to the aneurysm, e.g. through the aneurysm neck.

The method of these examples may comprise performing a first computational fluid dynamics simulation of the blood flow within the patient vascular structure before deployment of an implantable medical device; simulating the deployed configuration of an implantable medical device within the target location′ performing a second computational fluid dynamics simulation of the blood flow within the patient vascular structure with the deployed implantable medical device; determining a change in blood flow in the patient's vascular structure between the first and second computational fluid dynamics simulations.

More specifically determining a change in blood flow in the patient's vascular structure between the first and second computational fluid dynamics simulations comprises determining one or both of maximum blood flow velocity and spatially-averaged blood flow velocity within the aneurysm for the first and second computational fluid dynamics simulations and determining a percentage blood flow reduction (blood flow velocity) within the aneurysm.

Alternatively, determining a change in blood flow in the patient's vascular structure between the first and second computational fluid dynamics simulations comprises: determining the blood flow rate (e.g. in m³/s) through the aneurysm neck for the first and second computational fluid dynamics simulations and determining the percentage reduction in blood flow rate in the second simulation relative to the first simulation.

The flow diversion index may indicate a better fitting device where the percentage blood flow reduction is above a threshold value or within a target range. For example the flow diversion index may take a value between 0 and 1 where 1 indicates optimum flow diversion. The flow diversion index may take a value of 1 where the percentage blood flow reduction is above a threshold value or within a target range and a value below 1 where the flow diversion percentage is outside of the optimal range.

The computational fluid dynamics simulation may comprise any known method allowing solution of the Navier-Stokes equations, for example Finite Volume, Finite Element, Spectral Element, Lattice Boltzman method.

Use of a suitability metric comprising a flow diversion index allows for the performance of a device to be simulated ahead of surgery to determine its performance. It allows for identification of a best performing device that might not be apparent based on other factors, such as sizing, alone. Use of a flow diversion index also allows for ranking of implantable medical devices of different types, for example flow diverting devices, such as stents, or intrasaccular and implantable neurovascular devices, to allow a medical practitioner to identify the optimal type of implantable medical device for a particular case.

In some examples, the suitability metric comprises a plurality of parameters selected from the dimension index, the apposition index, the landing zone index, the flow diversion index, the obstruction value and the vessel geometry value. Each parameter may take a value between 0 and 1, where 1 indicates a good fitting device and 0 a poor fitting device. The suitability metric may comprise a weighted sum of each of the selected parameters.

The weights applied to the constituent parameters of the suitability metric may be determined through a multiple regression (e.g. linear or polynomial) between the parameters (independent variables) and a measure of clinical outcome (dependent variable), for example the aneurysm occlusion success or complication rate.

In particularly preferable examples, the suitability metric comprises the apposition index and the landing zone index. These parameters together provide an accurate indication of a well-fitting device.

Preferably, the suitability metric comprises a neck protrusion value providing a measure of the distance that the deployed implantable medical device protrudes outside of a neck of an aneurysm of a vessel, into the vessel. The neck protrusion value indicating a better fitting device where it tends towards zero.

Preferably, the method for simulating the deployed configuration of the plurality of implantable medical devices at a target location within the three-dimensional model of the patient's vascular structure comprises simulating the deployed configuration of the plurality of implantable medical devices sequentially in order of unconstrained dimensions of the implantable medical device.

Preferably, the method of simulating the deployed configuration of the plurality of implantable medical devices at a target location within the three-dimensional model of the patient's vascular structure comprises extracting a centerline from the three-dimensional model of the patient's vascular structure, wherein the centerline is a middle axis of a vessel within the patient's vascular structure, and numerically simulating the expansion of the implantable medical device along the centerline within the three-dimensional model of the patient's vascular structure.

Preferably, the method of numerically simulating the expansion of the implantable medical device along the centerline within the three-dimensional model of the patient's vascular structure is based on one or more of the implantable medical device properties, geometrical constraints posed by the patient's vascular structure and forces applied to the implantable medical device by the patient's vascular structure.

Preferably, the method for simulating the deployment of a plurality of impantable intravascular devices to determine a best fit device for a target deployment location within a patient vascular structure further comprises determining a first selection of the plurality of implantable in implantable intravascular device based on a first suitability metric, and determining a best fit device within the first selection of implantable intravascular devices based on a second suitability metric.

By first narrowing down the plurality of implantable intravascular devices to a first selection using a first suitability metric, which is then narrowed down further using a second suitability metric, the determination of a best fit device can be made more computationally efficient. In particular, the calculation of the first suitability metric may be less computationally intensive than the calculation of the second suitability metric. For example, the first suitability metric may comprise the dimension index and the second suitability metric may comprise one or more of the apposition index, the landing zone index, the porosity index and flow diversion index.

Determining a first selection of the plurality of implantable medical devices based on a first suitability metric may comprise: simulating the deployed configuration of the plurality of implantable medical devices and determining the difference between a dimension of the implantable medical device in the deployed configuration and a corresponding dimension of the target location, and determining a first selection of the plurality of implantable medical devices in which the difference between the dimension of the implantable medical device in the deployed configuration and the dimension of the target location is below a threshold.

Where the target location comprises a vessel portion having a target length, the dimension of the implantable device may be the length of the device in the deployed configuration and the corresponding dimension of the target location is the target length of the vessel portion.

The method may comprise simulating the deployed configuration of the plurality of implantable medical devices sequentially in order of the unconstrained dimension of the implantable medical device, preferably in order of increasing unconstrained dimension. In particular, where the target location comprises a vessel portion having a target length, simulating the deployed configuration of the plurality of implantable medical devices comprises: simulating the deployed configuration of the plurality of implantable medical devices sequentially in order of increasing unconstrained length of the implantable medical device. In an example in which the unconstrained dimension is unconstrained length, the method may comprise selecting a first device having an unconstrained length that is below the length of the target location; simulating the deployed configuration of the first device and comparing the deployed length of the device to the target length; where the deployed length is less than the target length, selecting a second device having an increased unconstrained length compared to the first device and repeating these steps until a selected device is determined to have a deployed within a target length range based on the target length. The target length range may be centered on the target length or the target length may be the lower bound of the target length range. The method preferably then comprises determining the second suitability metric for one or more selected devices having a deployed length within the correct range. This process may be used for other dimensions, for example the diameter of an intrasaccular device or for the dimensions of a deployed implantable device relative to the corresponding dimensions of the target location, where the coordinates may be defined relative to the patient's anatomy.

By simulating the deployed configuration of the plurality of implantable intravascular devices sequentially from the shortest to longest device (or from a shortest dimension to longest dimension of the device more generally), the simulating process is made more computationally efficient. Particularly because the whole device does not have to be fully re-simulated each time, only the addition of a small additional length to the device may need to be simulated. This subsequently reduces the computational burden of the simulation of a plurality of devices.

The method may additionally comprise, prior to simulating the deployed configuration of the plurality of implantable medical devices, the plurality of implantable medical devices are selected by: determining the maximum vessel diameter in the target location of the three-dimensional model, excluding a portion of the target location corresponding to an aneurysm (where the position of the aneurysm may be determined as defined above); accessing a database of candidate devices and selecting the plurality of implantable medical devices to be simulated as the implantable medical devices within the database having an unconstrained diameter within a target range based on the maximum vessel diameter. This provides an additional step for filtering a large number of candidate devices by first selecting by diameter size. In this step no simulation is required, since the devices are selected based on the properties and morphology of the vascular structure and the unconstrained diameter of the candidate devices. Therefore the process of determining a best fit device from a large number of candidate devices is made more computationally efficient.

Determining a best fit device within the first selection of implantable medical devices based on a second suitability metric may be determined using the method of determining a suitability metric as described above. In particular, the second suitability metric may comprise one or more of apposition index, landing zone index, porosity, flow diversion, obstruction value, vessel geometry value. In preferable examples the method comprises determining a selection of the plurality of implantable medical devices based on the correspondence of a deployed dimension relative to a corresponding dimension of a target location and then determining the apposition index for each of the selected implantable medical devices to determine a best fit device.

Optionally, the method for simulating the deployment of a plurality of implantable medical devices to determine a best fit device for a target deployment location within a patient vascular structure further comprises ranking the plurality of simulated implantable medical devices based on the suitability metric.

Optionally, the plurality of implantable medical devices comprise stents, such as braided stents. Alternatively, the plurality of implantable medical devices comprise implantable intravascular devices, intrasaccular devices, flow-diverting devices, or implantable neurovascular medical devices. Implantable neurovascular medical devices may include, for example, stents such as arterial stents, intracranial stents, or carotid stents, embolic coils, aneurysm coils, flow diverters, embolic protection devices, or neurothrombectomy devices. Devices or tools used when implanting implantable medical devices, such as catheters, balloon systems, guidewires, or stent retrievers may be additionally simulated when simulating the deployed configuration of a plurality of implantable medical devices at a target location. Said devices or tools may be simulated to simulate their use when implanting an implantable medical device or their use when withdrawing from implanting an implantable medical device. Alternatively or additionally, said devices or tools may be simulated to assist in the simulation of the deployment of implantable medical devices.

In some examples the plurality of devices comprise a plurality of implantable device types, for example flow diverting devices and intrasaccular devices or two or more of any of the implantable medical devices mentioned herein. The method may comprise determining a suitability metric for different implantable device types and therefore providing a best fit device type for a particular application. In these examples, preferably the suitability metric comprises the flow diversion metric and the method selects a best fit device, from the plurality of implantable device types, based on the suitability metric.

According to a second aspect of the present invention, a server is configured to perform the method of the first aspect of the present invention and its optional features.

According to a third aspect of the present invention, a non-transitory computer-readable medium comprising instructions which, when executed by a processor, cause the processor to perform the method of the first aspect of the present invention and its optional features.

### BRIEF DESCRIPTION OF THE DRAWINGS

Embodiments of the present invention are described below, by way of example only, with reference to the accompanying drawings, in which:

FIG. 1 illustrates in schematic form a system suitable for implementing aspects of the invention.

FIG. 2 is a flow diagram setting out the method for simulating the deployment of a plurality of implantable medical devices, performed by the system according to an embodiment.

FIG. 3 is a flow diagram setting out a method for creating a three-dimensional model, performed by the system according to an embodiment.

FIG. 4a shows a user interface for the selection of a region of interest according to an embodiment.

FIG. 4b illustrates an example of a three-dimensional model during the process of creating it, particularly during segmentation.

FIG. 4c illustrates an example of a three-dimensional model during the process of creating it, particularly during centerline extraction.

FIG. 4d illustrates an example of a three-dimensional model during the simulation of the deployed configuration of an implantable medical device.

FIG. 5a illustrates an example of a three-dimensional model during the determination of a measure of apposition between the implantable medical device in the deployed configuration and a wall of the vascular structure.

FIG. 5b illustrates an example of a three-dimensional model during the determination of a measure of apposition, particularly highlighting the aneurysm neck.

FIG. 6a illustrates an example saccular aneurysm within a vascular structure.

FIG. 6b illustrates an example of a three-dimensional model of a vascular structure marked with radial discrepancies.

FIG. 7a is a graph plotting the centerline radius and the cross-sectional area radius over length of the vascular structure.

FIG. 7b illustrates an example fusiform aneurysm within a vascular structure.

FIG. 8a illustrates an example implantable medical device deployed within vascular structure.

FIG. 8b illustrates a key for a sizing ratio weight function used in FIG. 8a.

FIGS. 9a and 9b illustrate an example of a model of a vascular structure marked with sizing requirements for an implantable medical device.

FIG. 10 is a table setting out example manufacturer recommendations for the deployment of a specific implantable medical device model within a patient's vascular structure.

FIG. 11 shows a user interface for the selection of an implantable medical device for simulation according to an embodiment.

### DETAILED DESCRIPTION

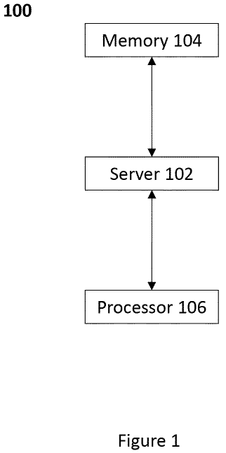

FIG. 1 shows a block diagram of a system 100 suitable for implementing embodiments of the invention. System 100 includes a server 102 that is communicative coupled to a memory device 104 and a processor 106.

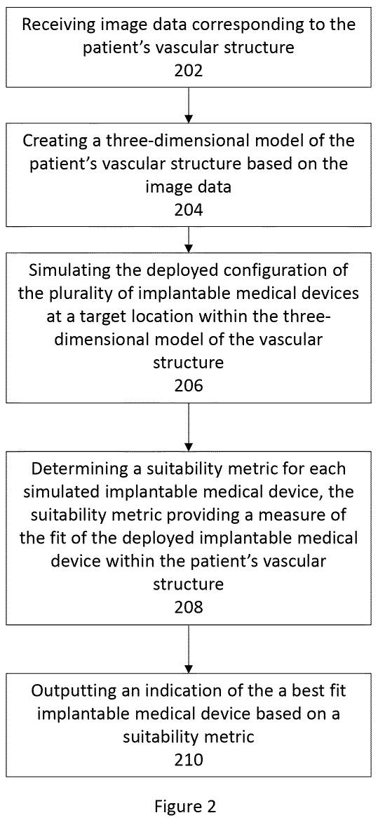

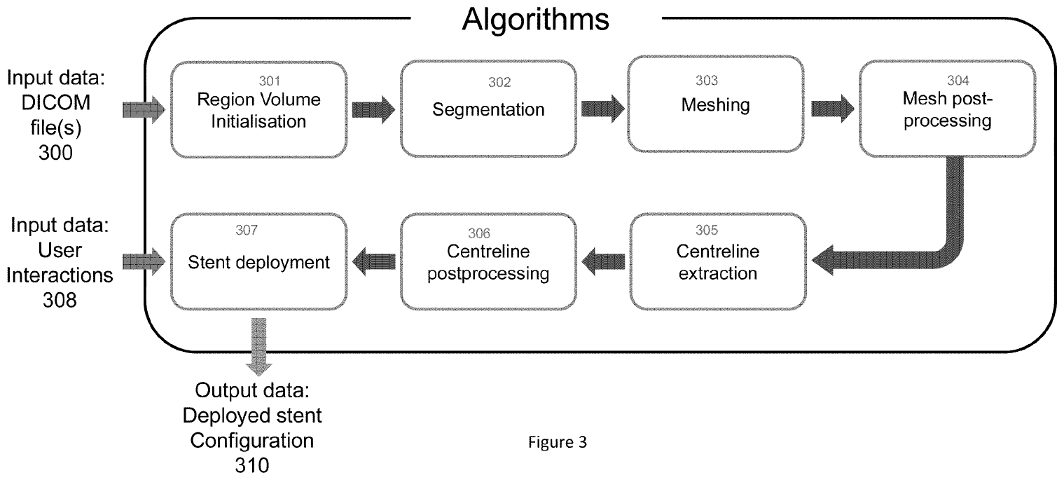

FIG. 2 sets out a method for simulating the deployment of a plurality of implantable medical devices to determine a best fit device for a target deployment within a patient's vascular structure. The method of FIG. 2 may be performed by any processor configured to execute the defined method steps, such as the system described above. FIG. 3 is flow diagram indicating the algorithms used in creating a three-dimensional model of a patient's vascular structure and simulating the deployed position of an implantable medical device, in this case a stent. Steps 300 to 306 correspond to steps 202 and 204 of the method according to the present invention illustrated in FIG. 2. Simulation of the stent deployment (step 307 of FIG. 3) is then repeated so as to simulate the deployed position of a plurality of implantable medical devices, where a suitability metric is calculated for each to determine a best fit device.

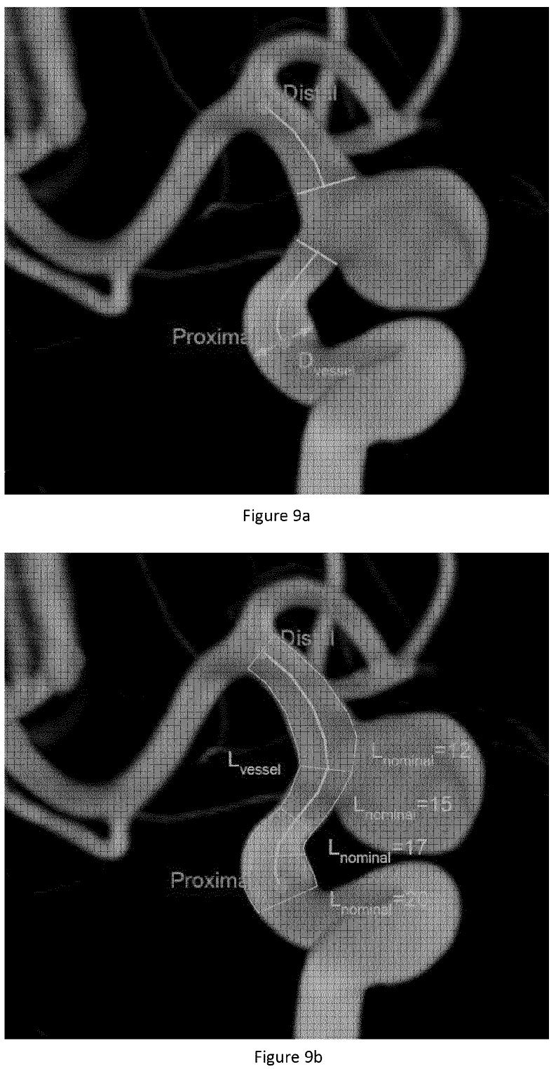

The plurality of implantable medical devices to be simulated may be acquired from a database of candidate devices and their associated manufacturer's guidance. The database may be stored on and retrieved from the memory device 104. The database may include a number of different models of candidate devices manufactured by different manufacturers and for each of these, a range of different sizes (e.g., different diameters and different lengths), each of which have a recommended vessel deployment diameter range, specified in the manufacturer's guidance. The associated manufacturer's guidance may include the size of the device required for a particular sized vessel, as well as the relationship between the length of the device after deployment and its diameter. It is possible to obtain an estimate of the deployed length of a device by assuming that the expansion of the device is limited by the diameter of the vessel. However, this is an oversimplification that assumes that the vessel has a regular, circular cross section, which it may not. Therefore, in one embodiment, the manufacturer's guidance may be used to select, from each manufacturer's model, an initial plurality of devices with the smallest diameter that is greater than the largest vessel diameter in the target location, to simulate before sequentially working through the length sizes. FIG. 10 is an example table setting out manufacturers' recommendations for the deployment of a specific implantable medical device model within a patient's vascular structure.

In step 202, image data corresponding to the patient's vascular structure is received. The image data may be a series of 2D images of the patient's vascular structure, which may be loaded in the form of a single-frame or multi-frame Digital Imaging and Communications in Medicine (DICOM) image, as shown at 300 in FIG. 3. The image data may be received by a processor 106 from a memory device 104. The image data may be stored on a memory device 104 which is communicatively coupled to the processor 106 in order to transfer the image data to the processor 106, e.g., via a public network such as the internet, or a private network or virtual private network, or data bus.

Optionally, a Maximum Intensity Projection (MIP) model, volume rendering, or other three-dimensional rendering of the patient's vascular structure may be created based on the image data before a full three-dimensional reconstruction of the vascular geometry and proceeding to step 204. These three-dimensional rendering techniques may be used to show the content of the image data before the actual reconstructions of the vascular geometry. When a MIP model or volume rendering has been created, it may be rotated, moved and/or enlarged using user inputs, e.g., mouse, trackpad, keyboard, to enable a user to view all aspects of the model and to select a region of interest.

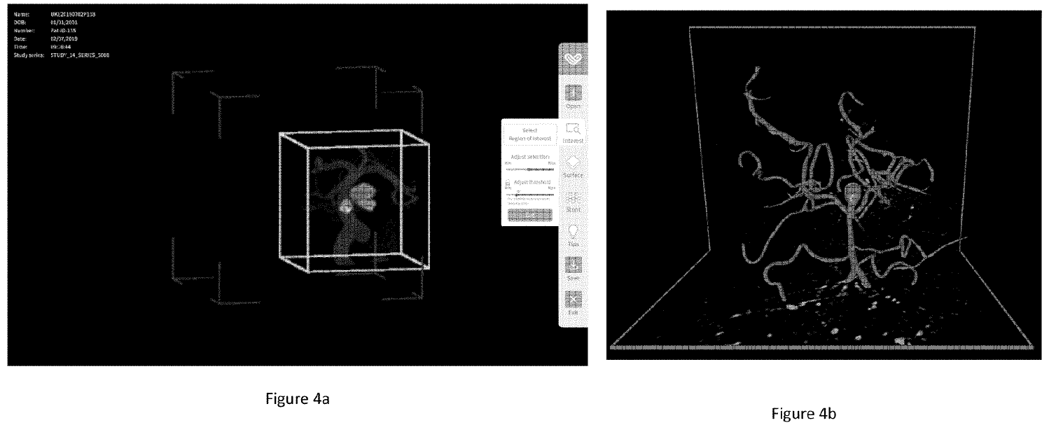

In step 301 of FIG. 3 region volume initialisation is performed. In this step a region of interest is selected. The selection may be made by a user using a user interface to manually define a region of interest or, alternatively, the region of interest may be automatically selected by the software. FIG. 4a shows an example user interface for the selection of a region of interest by a user. The user interface shows the details of the image data file in the top-left corner, including the patient's identification number for the image data and a time and date at which the image data was taken. The user interface further shows a three-dimensional box superimposed onto the initial three-dimensional rendering of the vascular geometry. This three-dimensional box is the selection areas for the region of interest and may be rotated, moved and/or enlarged using user inputs, e.g., mouse, trackpad, keyboard, to enable a user to view all aspects of the model and to select the appropriate region of interest. The user interface further includes a widget on the right-hand side which includes sliding bars to adjust the selection size of the region of interest.

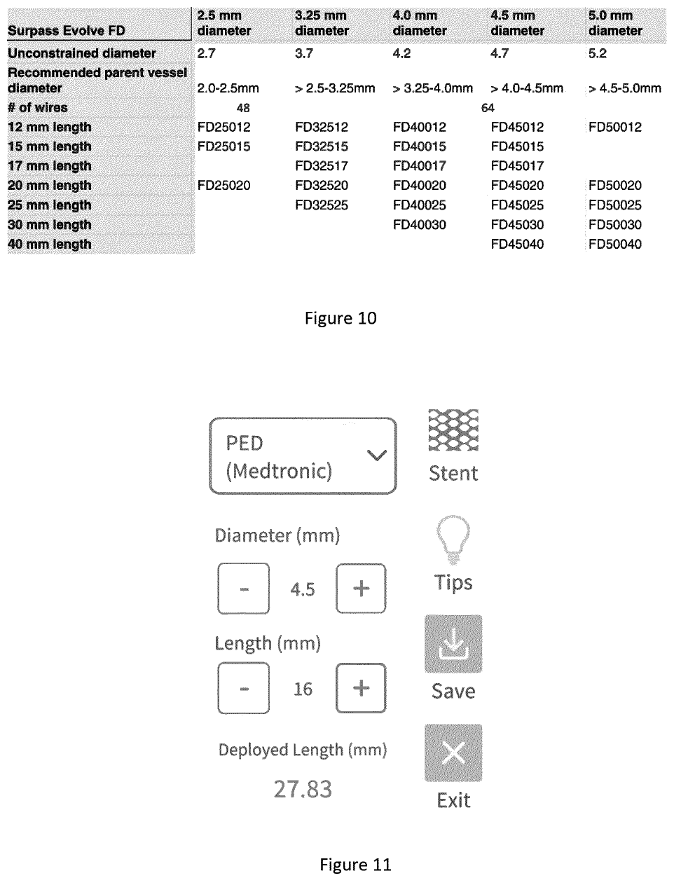

The user interface further includes a banner menu positioned down the right-hand side, which includes multiple buttons for the user to: select options such as opening a new set of image data, selecting a region of interest, selecting viewing options for the model, selecting a stent for deployment, tips for assistance with the program, save the model, or exit the program. If the option of selecting a stent for deployment is selected in the user interface the widget of FIG. 11 is displayed, which allows the user to select the manufacturer's model of the device, the diameter of the device, and the length of the device and displays the deployed length of the device as it is simulated. As each of these are changed by the user, the corresponding display of the simulated device, such as that illustrated in FIG. 4d, is changed.

The region of interest is the selected region within the patient's vascular structure in which the implantable medical device may be deployed, for example the location of an aneurysm. By focusing only on the region of interest within the patient's vascular structure, rather than the whole imaged region, the amount of data to process is reduced, thereby reducing the computational cost and speeding up the remaining steps of the method. As described above, rather than receive a user selection of the region of interest, for example by the provision of a UI with a ROI selection tool such as that described above, the region of interest may be automatically determined by the software. For example, the software may search the image to determine the location of an aneurysm (for example using one of the techniques described below) and automatically define the ROI around the located aneurysm. Where multiple possible aneurysms or regions of interest are identified by the software, the software may present the user a number of candidate ROIs such that the user can select the intended ROI for the further processing steps of the method.

Where the region of interest is user-selected, the model may be viewed in different display options in order to assist in the selection of the region of interest. For example, a shell mode (or hourglass mode) in which only the shell of the vascular structure is shown (along with a centerline of the vascular structure as will be described below), or an opaque mode in which the vascular structure is opaque in order to obtain a clear image of the general vascular structure. When a region of interest has been selected, the method may proceed to step 204.

In step 204, a three-dimensional model of the patient's vascular structure is created based on the image data. The three-dimensional model may be created by extracting clinically relevant information about the patient's vascular structure from the image data, which is used to reconstruct a three-dimensional model of the vascular geometry in order to assist with pre-operative planning. The three-dimensional model may additionally display a centerline of the patient's vascular structure. The centerline represents the middle axis of a vessel within the vascular structure and may be extracted as described below. A centerline radius provides a measure of the general radius of the vessel. It may be calculated in a number of ways. For example it may be defined as the minimum distance from a point on the centerline to the nearest point on a wall of the patient's vessel within a region of interest. It may also be defined as the average or maximum distance from a point on the centerline to the nearest point on the vessel wall, in that circumference/cross section.

The creation of the three-dimensional model may be carried out by segmenting a volume rendering of the image data into separate parts of the volume corresponding to, for example, blood vessels and aneurysms as foreground, and the remaining volume as background, as set out at step 302 of FIG. 3 and as shown by FIG. 4b. The separate parts of the volume may be labeled as such and may be identified as such by the blood vessels and aneurysms containing contrast that was used during the imaging process. FIG. 4b is an example three-dimensional model that shows this separation of the blood vessels and aneurysms as foreground by coloring them, whereas the remaining volume is designated as the background. This example view is in an opaque mode in which the vascular structure is opaque in order to obtain a clear image of the general vascular structure. This example shows clearly the benefit of segmenting the rendering in this way, so that the patient's vascular structure can be easily identified. Automated segmentation of the vascular structure can be obtained by one of the following methods: thresholding, region growing and deformable models such as active contour and level set methods.

The creation of the three-dimensional model is further carried out by meshing, as set out at step 303 of FIG. 3, in order to convert the labels produced during the segmentation into a surface mesh of the patient's vascular structure.

This step consists in creating a polygonal surface representation of an iso-surface through a three-dimensional scalar field sampled on a rectangular grid, i.e., the DICOM image. A marching cube algorithm may be used for meshing. A marching cube algorithm is an iterative algorithm for creating surfaces from a three-dimensional scalar field. In one embodiment, the three-dimensional scalar field is the intensity of the original DICOM image or a derived function, e.g., a level-set function obtained from level set segmentation method.

Next, post-processing is applied to the generated surface mesh in order to simplify and clean-up the surface to produce a good quality mesh, as set out at 304 of FIG. 3. Mesh post-processing may include the steps of identifying the largest connected component (e.g., using graph theory, depth-first search, and discarding small disconnected components), mesh simplification to reduce the number of triangles and simplify the mesh (e.g., using an edge collapse method), and mesh smoothing to regularize and remove noise from the surface of the mesh (e.g., using Laplacian smoothing or another algorithm suitable to smooth a polygonal mesh).

The creation of the three-dimensional model may further require centerline extraction, as set out at 305 of FIG. 3 and as shown by FIG. 4c. The centerline may, for example, represent the middle axis of a vascular structure providing a reduced representation of blood vessels and a means to guide the simulated deployment of an implantable medical device. The extraction of the centerline may be carried out as described in Antiga, L., & Remuzzi, A. (2002); Patient-Specific Modeling of Geometry and Blood Flow in Large Arteries, which requires the specification of source and target seed points placed at the vessel's terminals, which may be selected manually or in a fully automated manner. FIG. 4c is an example three-dimensional model which shows the extracted centerline as a line representing the middle axis of a vascular structure, with a transparent shell rendering of the vascular structure itself. This example view is in a shell mode in which only the shell of the vascular structure is shown along with a centerline of the vascular structure. The extracted centerline may be further submitted to post-processing, as set out at 306 of FIG. 3. Point coordinates and associated radii may be regularized through smoothing and re-interpolation.

In step 206 and step 307, the deployed configuration of a plurality of implantable medical devices at a target location within the three-dimensional model of the vascular structure is simulated. The simulation may comprise a mechanical and/or geometrical simulation of the expansion of the device. The simulation of step 206 and 307 may comprise performing a numerical simulation of the expansion of the implantable medical device based on one or more properties of the implantable medical device and one or more properties of the patient's vascular structure. The one or more properties of the implantable medical device may comprise mechanical properties of the device, geometrical constraints of the device, or unconstrained dimensions of the device, such as length, volume, thickness, diameter, radius, or height. The one or more properties of the patient's vascular structure may comprise geometrical constraints of the structure, dimensions of the structure, or local morphology of the structure. The numerical simulation may be carried out as described in EP3025638—METHOD FOR DETERMINING THE FINAL LENGTH OF STENTS BEFORE THE POSITIONING THEREOF or a method based on Finite Element Models as described in Ma, D., Dumont, T. M., Kosukegawa, H. et al. High Fidelity Virtual Stenting (HiFiVS) for Intracranial Aneurysm Flow Diversion: In Vitro and In Silico. Ann Biomed Eng 41, 2143-2156 (2013), or other simulation techniques. The numerical simulation may comprise simulating the expansion of the implantable medical device with constraints imposed by the geometry of the patient's vascular structure. The simulation may comprise simulating the forces applied to the implantable medical device by the patient's vascular structure and, optionally, a delivery system, device or tool used to deploy or position the device. The method may comprise numerically simulating the expanded configuration of the implantable medical device within the constraints imposed by the patient's vascular geometry as defined in the three-dimensional model, where the numerical simulation is preferably also based on mechanical constraints of the implantable medical device, for example the braiding of the wires of the device.

In some examples simulating the deployed configuration of the implantable medical device may comprise the following steps:
h. determine a ratio indicating the change in length of the stent as a function of the local morphology of the vascular structure
i. obtain the three-dimensional centreline from the three dimensional model of the vascular structure;
j. define the location of the starting point at which the device will be placed in the three-dimensional model of the vascular structure;
k. divide the centreline of the vascular structure into a plurality of segments;
I. determine the descriptive parameters of the morphology of said vascular structure for the first of the plurality of segments, where the descriptive parameters comprise geometrical parameters and/or mechanical factors;
m. calculate the length of the stent for said first segment using the indicator ratio of step a)
n. subtract said length of the segment calculated in step f) from the nominal length of the stent in order to obtain a new nominal length; if said new nominal length is different from 0 then steps e) to g) will be repeated for the segment contiguous with the preceding segment; if the new nominal length is approximately 0, all the distances of each segment will be added together, and this sum will be the final length of said stent after its positioning.

The ratio of step a) may be determined based on mathematical modeling.

At step 310 the deployed stent configuration may be outputted. FIG. 4c is an example three-dimensional model which shows the extracted centerline as a line representing the middle axis of a vascular structure, with a transparent shell rendering of the vascular structure itself and a simulated deployed implantable medical device shown as a mesh.

The method requires the defining of a target location within the vascular structure at which the implantable medical device is to be deployed during the simulation. The nature and definition of the target location will depend on the specific type of device and how it is to be deployed. For example, for a stent or other type of flow diverting device, the target location is likely to be a portion of vessel within the vascular structure defined by a start and end position (such that the vessel portion has a length defined as the length of the vessel portion between the start and end position). These may be defined as distal and proximal positions, based on their location relative to an entry location into which the device is inserted into the body (or relative to the blood flow in the vessel). In contrast, an intrasaccular device is intended to expand within an aneurysm and so the target location may not be defined as a length of vessel between a start and end position but as the volume of an aneurysm.

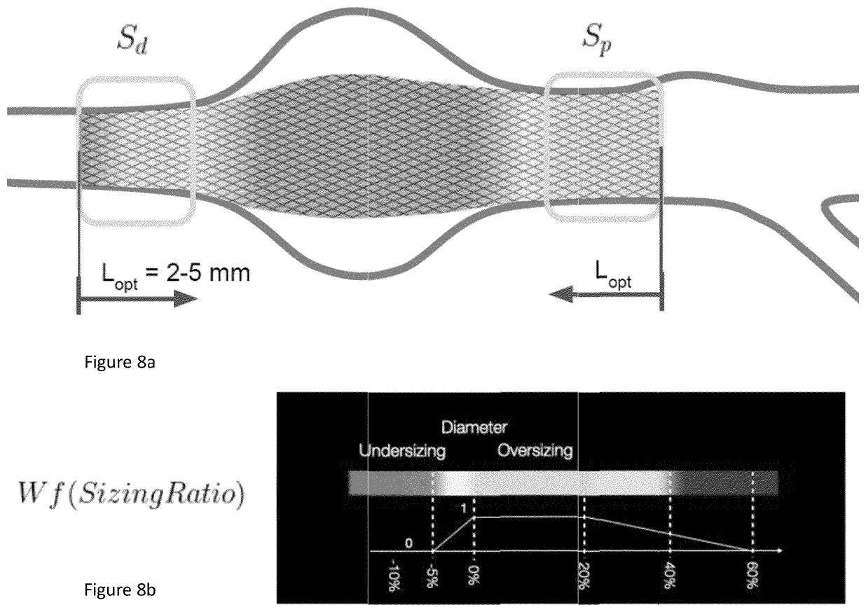

In the present example, in which the simulation of a stent is illustrated, the target location is defined as a portion of the vessel within the vascular structure extending between a start and end position. In the example of the figures, these are referred to as distal and proximal positions. FIG. 4d shows a distal position on the centerline marked “D” and a proximal position on the centerline marked “P”. FIGS. 9a and 9b further illustrate a distal position on the centerline marked “Distal” and a proximal position on the centerline marked “Proximal”. The target location of this example is a portion of the vascular structure having a target length between the distal position and a proximal position.

This type of target location, corresponding to a length of vessel containing an aneurysm may be defined in alternative ways. For example, the target location may be defined as a portion of the vascular structure extending distally and proximally from an identified aneurysm by a predetermined length. In some examples, the distal position and the proximal position (alternatively, the start and end positions) may be automatically selected based on a recommended length from the location of an aneurysm selected by a user using the user interface. Alternatively, the aneurysm may alternatively be automatically detected, as described below, and a recommended length from the detected aneurysm may be used to automatically select the distal position and the proximal position.

In the present example, the distal position and the proximal position may be selected by a user and marked on the centerline of the three-dimensional model. This allows the user to select the length of the vessel over which the device should be deployed. A target length should be long enough to ensure that the implantable medical device is not too short after deployment, so it may sufficiently fulfill its intended purpose. The target length should be short enough to ensure that flows which are not intended to be obstructed by the implantable medical device are not obstructed.

The deployed length and deployed configuration of an implantable medical device can be obtained through a numerical simulation of the expansion of the implantable medical device within the three-dimensional model of the patient's vascular structure, represented by the centerline. In particular, the deployed configuration is simulated based one or more properties of the implantable medical device and/or one or more properties of the patient's vascular structure, such as the geometrical constraints posed by the patient's vascular structure and/or forces applied to the implantable medical device by the patient's vascular structure and, optionally, the delivery system. The numerical simulation may be carried out as described in EP3025638—METHOD FOR DETERMINING THE FINAL LENGTH OF STENTS BEFORE THE POSITIONING THEREOF or with a more computationally demanding method based on Finite Element Models as described in Ma, D., Dumont, T. M., Kosukegawa, H. et al. High Fidelity Virtual Stenting (HiFiVS) for Intracranial Aneurysm Flow Diversion: In Vitro and In Silico. Ann Biomed Eng 41, 2143-2156 (2013) or other simulation techniques.

Using this method, the deployed dimensions of the device, such as the length, volume, thickness, diameter, radius, or height of the device, may be determined by simulation without the need of calculating the full deployed configuration of the implantable medical device, which can be determined at a later stage, if needed. In this way a two-step simulation may be carried out in which an initial simulation, with a lesser computational requirement, may be first carried out for a wider range of devices to determine one or more deployed dimensions of the devices. Then a second, more computationally intensive, simulation step may be carried out for a selected number of the initial devices in which a full simulation of the final configuration is carried out.

When the deployed configuration of the plurality of implantable medical devices is simulated, the device may be positioned starting from the distal position of the target location, extending towards the proximal position.

Optionally, the simulated deployed configuration of each of the plurality of implantable medical devices within a three-dimensional model of the vascular structure may be displayed to a user. The simulated deployed configuration may be displayed to the user as shown in FIG. 4d.

In step 208, a suitability metric for each simulated implantable medical device is determined. The suitability metric provides a measure of the fit of the deployed implantable medical device within the target location of the patient's vascular structure. The suitability metric may optionally take into account the manufacturer recommendations for the deployment of the implantable medical device within a patient's vascular structure, such as recommended stent models, lengths or diameters.

FIG. 10 is a table setting out example manufacturer recommendations for the deployment of a specific implantable medical device model within a patient's vascular structure. The table includes the different diameter options for the device model, with corresponding unconstrained diameters, corresponding recommended vessel diameter, and corresponding number of wires in the mesh of the device. The table further includes the different length options for the device model at each different diameter option, and the corresponding device model number for each option. FIG. 9a further illustrates the vessel diameter (Dᵥₑₛₛₑᵢ) at the proximal point of the target location.

The suitability metric may include and take into account multiple different parameters, as described below. The computer implemented method outputs an indication of the best fit device according to the device that scores highest according to the suitability metric. Optionally, in some examples the user may be able to weight the different parameters of the suitability metric, such that the suitability metric promotes devices with particularly essential metrics to a specific scenario. In other examples the weighting of different constituent parameters of the suitability metric is performed automatically, for example based on the type of implantable medical device and the target location.

The suitability metric may include a measure of the correspondence between one or more dimensions of the implantable medical device in the deployed configuration (as predicted by the simulation) and the corresponding dimensions of the target location. For example, where the target location is a longitudinal portion of vessel defined between a distal and proximal point (which may be user selected or automatically selected to define the target location), the suitability metric may provide a measure of the discrepancy between the length of the deployed device determined by the simulation and the length of the vessel between the distal and proximal point. Where the device is an intrasaccular device the dimension may be a radius or diameter of the deployed device, which is compared to a measured diameter of the aneurysm. Alternatively dimensions may be determined for the deployed device and compared to those of the aneurysm. The dimensions may comprise length, volume, thickness, diameter, or other such dimensions. The suitability metric is dependent on the measure of the correspondence such that a closer match between the simulated deployed dimension and corresponding target dimension indicates a better match.

The measure of the correspondence of the length of the implantable medical device and the length of the target location may be calculated using:

$$LengthDiscrepancy = \frac{abs ( {L}_{target} - {L}_{device} )}{{L}_{target}}$$

Where L_{target} is the length of the target location (i.e. a target vessel segment for deployment) and L_{device} is the deployed length of the device based on the simulation. Other device dimension correspondence can be determined in the same way and taken account within the suitability metric. The dimension index may be defined as 1—DimensionDiscrepancy as defined above to provide a value of 1 for an exactly fitting device. Alternatively a weight function may be used to assign a value of 1 where the dimension is within a target range based on the target dimension.

The suitability metric may additionally or alternatively include a measure of the apposition between the implantable medical device in the deployed configuration and a wall of the vascular structure. The apposition measures how close two items are to each other, for example, a specific point on the implantable medical device in the deployed configuration and the nearest point on the wall of the vascular structure. In an example scenario in which an implantable medical device is being used for aneurysm healing, incomplete apposition may result in residual blood flow to the aneurysm which can affect aneurysm occlusion and reduce aneurysm healing. Malapposition is also associated with adverse events such as thrombo-embolic complications and implantable medical device migration.

The measure of the apposition includes a measure of the distance between an outer surface of the implantable medical device in the deployed configuration and the wall of the vascular structure. A plurality of points on the outer surface of the implantable medical device in the deployed configuration may be measured to the corresponding nearest point on the wall of the vascular structure, for each of the plurality of points on the outer surface of the implantable medical device.

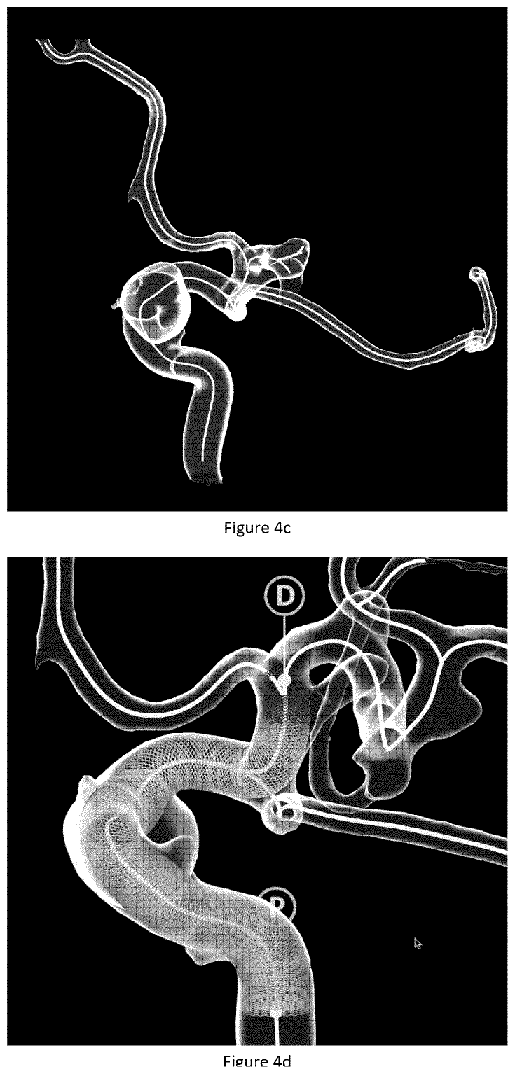

The measure of the apposition may include an apposition index comprising a percentage of the surface of the implantable medical device that is within a threshold distance from the wall of the vascular structure. FIG. 5a illustrates an example of a three-dimensional model which shows a transparent shell rendering of the vascular structure and a simulated deployed implantable medical device shown as a mesh. The mesh is shaded in different colors to display to a user areas of the deployed implantable medical device with good apposition and areas with malapposition.

Alternatively, the measure of apposition may include an apposition index comprising a percentage of the surface of the implantable medical device that is in contact with the wall of the vascular structure. In an example, the higher the percentage of the surface of the implantable medical device that is in contact with the wall of the vascular structure, the better the apposition and the better fitting the device is.

In the calculation of the apposition index, an area corresponding to an aneurysm neck should be excluded from this calculation, as the aneurysm neck by definition does not have good apposition. In particular, the surface area of the implantable medical device that is adjacent to the aneurysm neck should be excluded and the distance to the vessel wall from this portion of the surface area not used in the calculation of the apposition index. FIG. 5b illustrates an example of a three-dimensional model which shows a transparent shell rendering of the vascular structure and highlighted areas of malapposition in the aneurysm neck which should be excluded. Detection of a portion of the vascular structure corresponding to an aneurysm neck may be determined as described below.

For flow-diverting devices the aneurysm neck is viewed from within the vessel into a dome or the aneurysm. For intrasaccular devices the aneurysm neck is viewed from within a dome of the aneurysm into the vessel. In both embodiments, good coverage and low porosity are desired for the best fit. In the embodiment of intrasaccular devices, high apposition of the device is desired for best fit, so that the device blocks off as much of the entrance to the dome of the aneurysm as possible. The apposition of flow diverters at the neck of the aneurysm is not considered because the flow diverters do not enter the dome of the aneurysm (while the intrasaccular devices do)

The portion of a vessel corresponding may be determined by determining a portion of the vascular structure in the target location in which the radius of the vascular structure is anomalous. More specifically, the location of an aneurysm can be identified where the radius of a vessel within the model is much larger than the remainder of the vessel. These abrupt changes in the measured vessel radius can be used to identify the part of the vessel corresponding to the aneurysm and then the portion of the surface area of the deployed device which is directly adjacent to this part of the vessel can be excluded from the calculation.

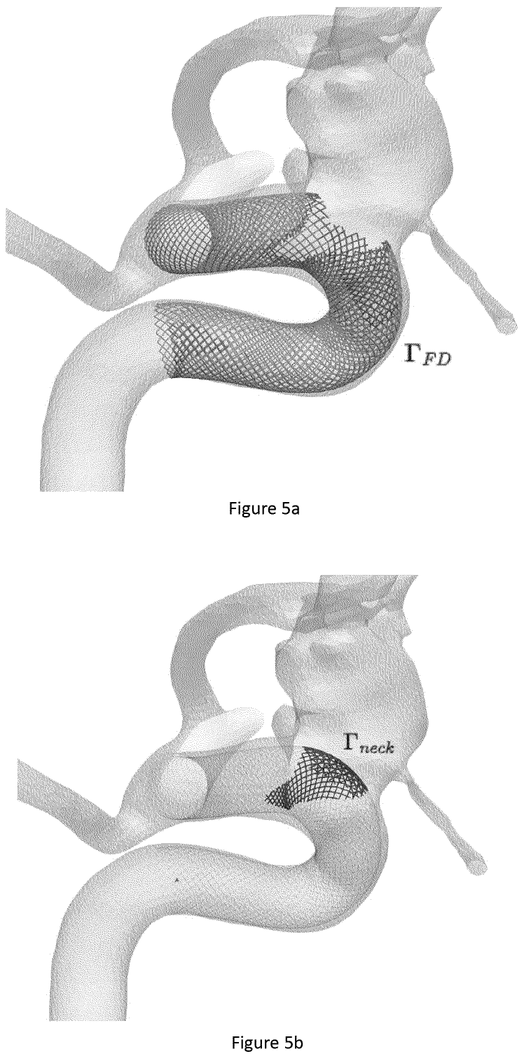

This kind of deviation in the radius can be determined in a number of ways. For example, the portion of the vessel corresponding to the aneurysm may be determined by determining a portion of the vascular structure in the target location in which the radius of the vascular structure is greater than an average of the remaining portion of the target location by beyond a threshold amount. Where there is a large discrepancy in the radius of the vascular structure, it may be assumed that a saccular aneurysm is located at this determined portion. FIG. 6a illustrates an example aneurysm within a vascular structure. The centerline of the vessel is shown by the dotted line, with the inner ring (centered on the dotted line) representing the centerline radius. As described above, the centerline radius provides a measure of the average radius of the vessel portion. One approximation is calculated by using the minimum distance between the centerline and vessel wall. The outer ring (which surrounds the inner ring) represents the actual radius of the vascular structure at the selected position in the model. Due to the large discrepancy in the centerline radius and the actual radius, it is clear that a saccular aneurysm is present.

FIG. 6b illustrates an example of a three-dimensional model with radial discrepancies illustrated by widening of the centerline of the vascular structure. The radial discrepancy illustrated is obtained using the following formula:

$$RadiusDiscrepancy = \frac{abs ( {R}_{centerline} - {R}_{cross - section} )}{{R}_{centerline}}$$

Where abs is the absolute value, R_{centeritne} is the centerline radius, and R_{cross-section} is the radius at the point a cross-section is being taken.

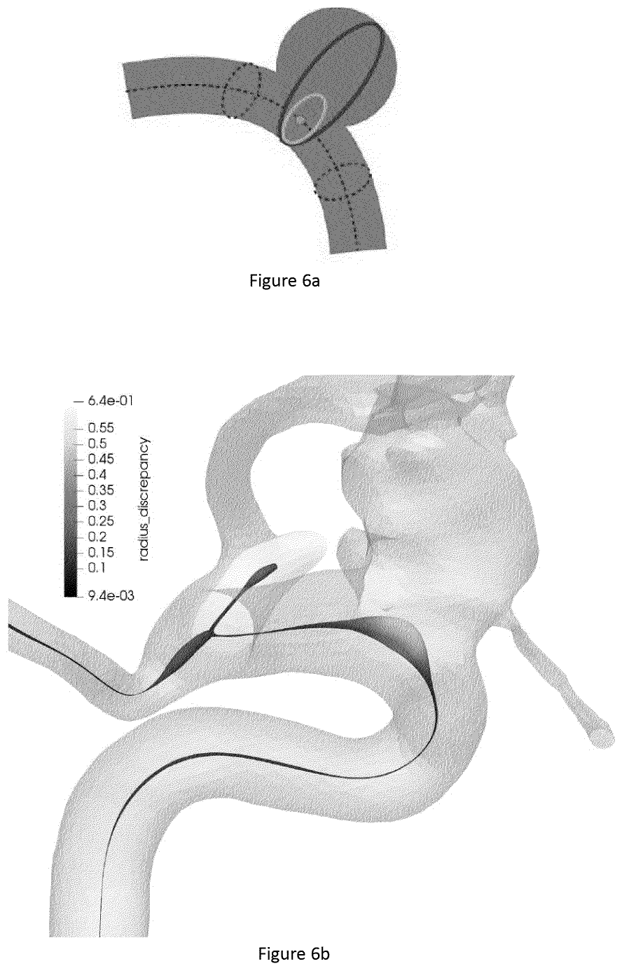

Alternatively, the portion may be determined by determining a portion of the vascular structure in the target location in which the cross-sectional area radius of the vascular structure is greater than the centerline radius of the remaining portion of the target location by beyond a threshold amount. Where there are large peaks in the radius of the vascular structure, it may be assumed that a fusiform aneurysm is located at this determined portion, as normal physiological arteries taper slowly in the distal direction. FIG. 7a is a graph plotting the centerline radius and the cross-sectional area radius over a length of the vascular structure. The large peak in the cross-sectional area radius compared to the centerline radius shows that a fusiform aneurysm is located across the length of this peak. The vertical lines on the graph mark the limits of this detected fusiform aneurysm. FIG. 7b illustrates an example of a three-dimensional model which shows a transparent shell rendering of the vascular structure, a centerline, and a mesh which is shaded in different colors to show the cross-sectional area radius at each point on the centerline. This illustration shows a fusiform aneurysm within a vascular structure, marked by the change in shading of the mesh.

The apposition index may be calculated by excluding the portion of the outer surface of the implantable medical device corresponding to the determined portion of the vascular structure corresponding to the aneurysm. Finally, calculating the apposition index over the remainder of the outer surface of the implantable medical device.

As described above, FIGS. 5a and 5b illustrate the calculation of the apposition index for the case of a flow diverter device. The apposition index for the flow diverter device may be calculated using the following integral:

$$AI = \frac{1}{{S}_{FD} - {S}_{Neck}} {\int }_{{Γ}_{FD} - {Γ}_{neck}} GA ( x ) d Γ$$

Where: S_{FD} is the total surface area of the deployed flow diverter device; S_{Neck} is the surface area of the vessel corresponding to the aneurysm neck; Γ_{FD} is the surface area of the flow diverter device; Γ_{neck} is the surface area of the flow diverter device corresponding (i.e. adjacent to) the neck of the aneurysm; GA=1 when abs(d_{FD-watt})≤d_{threshold}; and GA=0 when abs(d_{FD-watt})≥d_{threshold}.

Optionally, the suitability metric includes a neck coverage value providing a measure of the percentage of the wall of the neck of the aneurysm covered by the implantable medical device, which is preferably an intrasaccular device in this embodiment. The wall of the neck may be classified as a region of the wall of the vascular structure which is within a threshold distance from an aneurysm neck. In an example, the higher the percentage of the neck wall of the aneurysm covered by the implantable medical device, the better fitting the device is. Alternatively, neck coverage value provides a measure of the percentage of the wall of the neck of the aneurysm which is in contact with or within a threshold distance of the implantable medical device, which is preferably an intrasaccular device is this embodiment. In an example, the higher the percentage of the neck wall of the aneurysm which is in contact with or within a threshold distance of the implantable medical device, the better fitting the device is.

Optionally, the suitability metric includes a neck protrusion value providing a measure of the distance that the implantable medical device protrudes outside of an aneurysm neck into a parent vessel. The neck protrusion value is preferably zero for the best fit and the closer to zero the neck protrusion value is, the better fitting the device is. In this embodiment, the implantable medical device is preferably an intrasaccular device.

The suitability metric may additionally or alternatively include a flow diversion index that provides a measure of the flow diversion achieved by the deployed device. The flow diversion index may indicate a better fitting device where a larger amount of flow diversion is achieved by the deployed implantable medical device. For example the flow diversion index may indicate optimal flow diversion where simulated flow diversion is above a threshold value or within a predefined range. In particular the flow diversion metric may provide a measure of the reduction in blood flow to an aneurysm achieved by a deployed implantable medical device.

To calculate the degree of flow diversion, the method may comprise comparing two computational fluid dynamics (CFD) simulations: a first simulation of blood flow within the patient vascular structure without any implanted device, which would serve as a baseline and another one with the implantable medical device deployed in its final configuration. Maximum velocity as well as spatially-averaged velocity (both calculated within the aneurysm) can then be used to calculate a percentage of flow reduction inside the aneurysm.

Another way to calculate the flow reduction could be accomplished by directly measuring the total flow rate (in m³/s) through the aneurysm neck (where the aneurysm neck may be located as described above in reference to the porosity index), and then calculating the percentage reduction between the simulations with and without device.

Any of the well-known CFD methods that allow solution of the Navier-Stokes equations (e.g., Finite Volume, Finite Element, Spectral Element, Lattice Boltzman method) may be used to carry out the two simulations mentioned above.

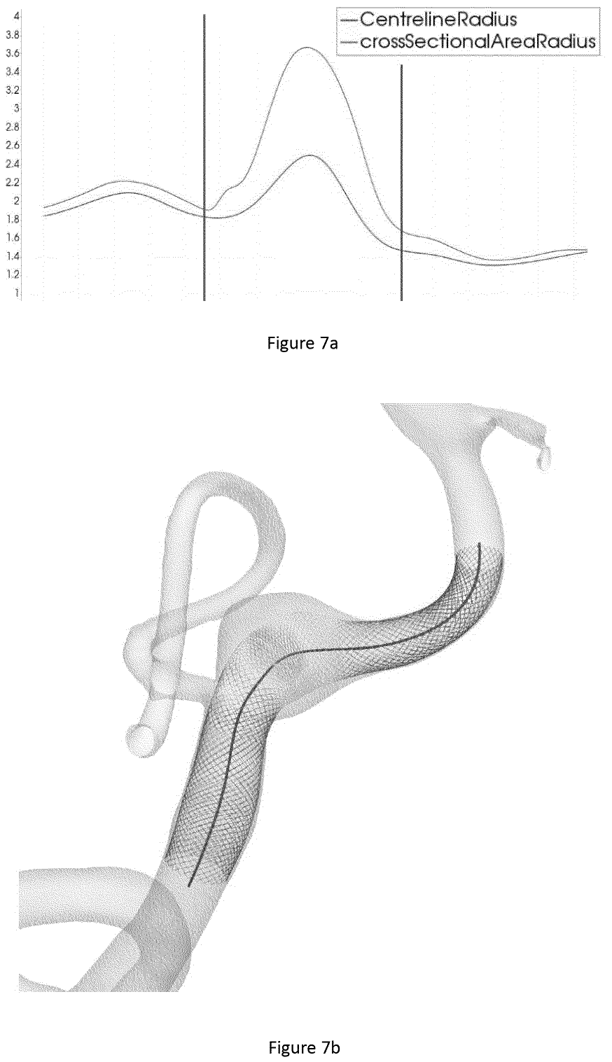

The suitability metric may additionally or alternatively include a landing zone index. The landing zone index includes a measure of the length of the landing zones of the implantable medical device. The landing zones include longitudinal sections of the deployed implantable medical device positioned immediately distally and proximally to an aneurysm neck in the target location, as labeled S_{d} and Sₚ in FIG. 8a. Preferably the distal and proximal landing zones are in straight vessel portions to ensure good apposition with the wall. The landing zone indicates a better fitting device where the landing zone length is above a threshold value.

When the landing zone length is above the threshold value, this ensures good purchase, good apposition and avoids device migration. FIG. 8a illustrates an example of a model which shows the outline of a vascular structure and a mesh representing a deployed implantable medical device which is shaded to show the sizing ratio weight function (shown in FIG. 8b) applied to each portion of the implantable medical device. The proximal landing zone is illustrated by the box marked Sₚ and the distal landing zone is illustrated by the box marked S_{d}. FIG. 8b illustrates the key for the shading used to show the sizing ratio weight function applied, as described below.

The apposition at the landing zones may be calculated by determining a sizing ratio using the ratio between the diameter of the implantable medical device and the diameter of the vessel at the landing zone. This sizing ratio assists in determining how well an implantable medical device is sized for that particular vessel segment.

The landing zone index may be calculated by using the below two integrals:

$${S}_{d} = \frac{1}{{L}_{opt}} {\int }_{0}^{{L}_{opt}} Wf ( SizingRatio ) ds$$

$${S}_{p} = \frac{1}{{L}_{opt}} {\int }_{{L}_{device} - {L}_{opt}}^{{L}_{device}} Wf ( SizingRatio ) ds$$

Where: Sₚ is the proximal landing zone sizing index as shown in FIG. 8a; S_{d} is the distal landing zone sizing index as shown in FIG. 8a; Lₒₚₜ is the optimal landing zone length for the particular implantable medical device (for example as indicated in the manufacturer's guidance) as shown in FIG. 8a; L_{device} is the length of the deployed implantable medical device; and Wf is a weight function, as shown in FIG. 8b. The landing zone sizing index, S, is the calculated according to:

$$S = \frac{{S}_{d} + {S}_{p}}{2} .$$

If the implantable medical device is well sized and long enough, S will tend towards 1. If the implantable medical device is poorly sized or the landing zone is too short, S will tend towards 0.

The weight function, Wƒ, can be used to penalize areas of the implantable medical device by assigning the area a weight lower than 1. FIG. 8b shows a key for a sizing ratio weight function used in FIG. 8a. In this example the weight function is structured as follows: The weight function assigns a weight of 0 where the implantable medical device has a deployed diameter that is more than 5% undersized relative to the diameter of the vessel in the landing zone. The weight function is a linearly increasing function between 5% and 0% undersized (i.e., where the device has a diameter which is smaller than the vessel diameter by between 0% and 5% of the vessel diameter), taking a value of 0 at 5% undersized and 1 where the diameters are perfectly matched. The weight function assigns a weight of 1 where the implantable medical device has a deployed diameter that is between 0% and 20% oversized based on the size of the vessel in the landing zone. The weight function is a linearly decreasing function between 20% and 60% oversized (i.e. where the deployed device has a diameter which is greater than the vessel diameter by between 20% and 60% of the vessel diameter), taking a value of 1 at 20% oversized and 0 at 60% oversized. The weight function applies a weight of 0 where the device is over 60% oversized. As above, this under or over sizing is determined based on the deployed diameter of the device relative to the local diameter of the vessel. The above weight function is just an example of a function that can be used to take into account the detrimental effect of undersizing or significantly oversizing devices. In general, where the deployed diameter corresponds with the vessel diameter or is slightly larger the metric should indicate a good fitting device.

The suitability metric may additionally or alternatively include a porosity index. The porosity index includes a measure of the porosity of the implantable medical device in the deployed configuration. Porosity is the ratio of the volume of voids in a mesh over the total volume of the mesh. The porosity of an implantable medical device may change as a mesh of the device changes shape and expands and contracts in different areas, depending on its deployed configuration. The porosity index may indicate a better fitting device where the porosity is below a threshold value in a selected portion of the outer surface of the deployed implantable medical device. The selected portion of the outer surface of the deployed implantable medical device may be a portion of the surface area corresponding with an aneurysm neck (i.e. Γ_{neck} in FIG. 5b). Low-porosity in the neck area can assist in speeding up aneurysm occlusion. The porosity may be controlled, for example, by applying more or less “push-pull” (i.e., forces applied by the user with the deployment system) during deployment to compact a device. Said “push-pull” forces may be applied by the user pushing or pulling the deployed implantable medical device with the deployment system to contract or expand the device in different areas. This may cause the density of the mesh of the device in different areas to change and therefore enable the porosity of the device to be controlled.

The porosity index may be calculated by determining a portion of the vascular structure corresponding to an aneurysm. The portion may be determined by determining a portion of the vascular structure in the target location in which the radius of the vascular structure is greater than an average of the remaining portion of the target location by beyond a threshold amount (or, more specifically, one of the methods described above in relation to the apposition index). Further, selecting the portion of the outer surface of the deployed implantable medical device corresponding to the determined portion of vascular structure corresponding to the aneurysm. Finally, determining the porosity of the selected portion of the outer surface of the deployed implantable medical device. This may be determined by simulating the configuration of the deployed implantable medical device and calculating the ratio of the volume of voids in a mesh over the total volume of the mesh. As above, a weight function may be applied to apply a value of 1 where the porosity is within an optimum range, a value of zero where the porosity is below a threshold value or above a threshold value and apply a linear function to apply a value varying between 0 and 1 where the porosity is just outside the optimum range, as described above with respect to the landing zone index.

The porosity index may alternatively be calculated by determining a portion of the vessel corresponding to the aneurysm neck and calculating the average of porosity across the cross-sectional area of the aneurysm neck opening. The lower the average of porosity across the cross-sectional area of the aneurysm neck opening, the better the fit of the device. For example, the most coverage of the aneurysm neck opening as possible is better for the fit of the device. In an example, the porosity of some intrasaccular devices is lower at the top/bottom surfaces, so ideally these low porosity surfaces are positioned perpendicular to the neck of the aneurysm, the more of the neck wall that is covered, the lower the porosity at the neck opening will be, and the device will have a better fit.

The suitability metric may additionally or alternatively include an obstruction value. The obstruction value provides a measure of the degree to which the deployed implantable medical device obstructs side branches within the three-dimensional model of the vascular structure. The obstruction value indicates a better fitting device where fewer side branches are obstructed and flow disturbances are limited. Shorter implantable medical devices will limit flow disturbances as much as possible and will also obstruct a lower number of side branches, which is preferable.

The suitability metric may additionally or alternatively include a vessel geometry value. The vessel geometry value provides a measure of the degree to which the deployed implantable medical device extends across one or more bends in the three-dimensional model of the vascular structure that comprise a radius of curvature below a threshold. The vessel geometry value indicates a better fitting device where fewer bends below the radius of curvature threshold are covered. Deploying implantable medical devices inside vessel bends is challenging and can result in poor apposition, as full expansion is more difficult when the implantable medical device is bent. Therefore, shorter deployed devices that cover fewer vessel bends are better fitting and hence preferable. As with each of the suitability metric parameters described herein, the vessel geometry value and the obstruction value may take a value between 1 and 0, 1 indicating a best fitting device and 0 indicating a poorly fitting device.

Optionally, a representation of the suitability metric or any one of its component parameters may be displayed on a corresponding simulated deployed configuration of each of the plurality implantable medical devices within a three-dimensional model of the vascular structure. The representation of the suitability metric may show the fit of the deployed implantable medical device at each point within the three-dimensional model. This may be displayed as a color range, with red areas marking areas of the deployed implantable medical device which do not have good fit or suitability and green areas marking areas of the deployed implantable medical device which do have good fit or suitability.

The suitability metric may be summarized as a numerical value and displayed next to the model for a device or a plurality of the devices. In particular, after simulating the plurality of implantable medical devices and determining the suitability metric, one or more of the devices may be displayed with the numerical value representing the suitability metric. The devices may be ranked and listed in order of fit based on the suitability metric.

In certain embodiments, each constituent parameter of the suitability metric takes a value between 0 and 1, where 1 indicates a good fit and 0 a poor fit. The suitability metric may be calculated based on a sum of the constituent parameters, which may optionally be weighted by a coefficient to select their relative importance in the calculation.

Optionally, based on this representation and as set out at step 308, the user may move the deployment position of the simulated implantable medical device and re-run the simulation to improve the suitability of an implantable medical device. In particular, in certain embodiments a user may adjust the start and end points (alternatively proximal and distal points) to change the target location in which the device is to be deployed. This may be achieved by dragging the start and end points (e.g., the P and D points displayed in FIG. 4D), which causes the simulation to re-run and the suitability metric to be recalculated. Alternatively, this may be done automatically where the suitability metric of a device may be improved by moving its deployment position. For example, the simulation may automatically vary the target location to determine a best fit location.

In step 210, an indication of a best fit implantable medical device based on the suitability metric is outputted. As described above, the suitability metric may include one or more of: a measure of the correspondence between the dimensions of the deployed device and the corresponding dimensions of the target location (a dimension index); a measure of apposition between the implantable medical device in the deployed configuration and a wall of the vascular structure (the apposition index); a landing zone index; a porosity index; a flow diversion index; an obstruction value; a neck coverage value; a neck protrusion value and a vessel geometry value. The indication of a best fit implantable medical device may include one or more identifiers of an implantable medical device, such as: a manufacturer of an implantable medical device, a manufacturer model of an implantable medical device, a diameter, a length, a material which the device is made of, or a positioning of the device within the vascular structure. The indication may alternatively be an indication of a plurality of best fit implantable medical devices, ranked based on their individual suitability metrics. The indication may be outputted to the user on a display in a form suitable for the user to interpret.

In an alternative embodiment, a first selection of the plurality of implantable medical devices is determined based on a first suitability metric. A best fit implantable medical device within the first selection of implantable medical devices is then determined based on a second suitability metric. This determination may be carried out using the determination methods described above, particularly those described in step 208 and step 210. An indication of this best fit implantable medical device based on a first suitability metric and a second suitability metric is outputted. The indication may be outputted to the user on a display in a form suitable for the user to interpret.

By first narrowing down the plurality of implantable medical devices to a first selection using a first suitability metric, which is then narrowed down further using a second suitability metric, the determination of a best fit device can be made more computationally efficient. In one example, the first suitability metric is a computationally simple calculation, such as determining an appropriate range of lengths and diameters for the implantable medical device or a measure of the correspondence between the length of the implantable medical device in the deployed configuration and the target length of the target location. The second suitability metric may subsequently include more computationally expensive calculations, such as one or more of a measure of apposition between the implantable medical device in the deployed configuration and a wall of the vascular structure; a landing zone index; a porosity index; a flow diversion index; an obstruction value; a neck coverage value; a neck protrusion value; and a vessel geometry value. Since the selection of implantable medical devices has already been narrowed down before it gets to the computationally expensive calculations, the method is made more computationally efficient. However, the first and second suitability metrics may be based on any one or more of the suitability metric parameters described above.

In one embodiment, the plurality of implantable medical devices to be simulated may be selected by firstly determining the maximum vessel diameter in the target location of the three-dimensional model, excluding a portion of the target location corresponding to an aneurysm. A database of candidate devices may then be accessed and the plurality of implantable medical devices is selected by determining the implantable medical devices within the database having an unconstrained diameter within a target range based on the maximum vessel diameter. FIG. 9a illustrates the vessel diameter at a proximal location within the vessel used to determine the target range. The target range may be defined by the manufacturers' guidance defining the device diameters that should be used for vessel diameter sizes, such as those shown in FIG. 10. Generally, the device diameter should be equal to or larger than the maximum vessel diameter. Therefore, the target range may have a minimum bound equal to the maximum vessel diameter and a maximum bound determined by an additional percentage of the maximum vessel diameter.

Preferably the selected plurality of implantable medical devices have the smallest unconstrained diameter which is above the maximum vessel diameter, to ensure good apposition. The selected plurality of implantable medical devices to be simulated may be expanded by sampling around the initially sized device in terms of diameter and length.

The determination of the first selection of the plurality of implantable medical devices based on a first suitability metric includes simulating the deployed configuration of the plurality of implantable medical devices and determining the difference between the length of the implantable medical device in the deployed configuration and the target length of the target location.

The simulating of the deployed configuration of the plurality of implantable medical devices may be carried out sequentially in order of the unconstrained length of the implantable medical device. In one example, the sequential order may be in order of increasing length from the shortest device with the selected diameter to the longest device with the selected diameter, until the deployed length of the device is longer than the vessel section. In another example, the sequential order may be in order of increasing length from a device with a length that is a nominal amount shorter than the target length, with the selected diameter, to the longest device with the selected diameter. By simulating the deployed configuration of the plurality of implantable medical devices sequentially from the shortest to longest device, the simulating process is made more computationally efficient. This is because the whole device does not have to be fully re-simulated each time, only the addition of a small additional length to the device may need to be simulated. This reduces the computational burden of the simulation of a plurality of devices. FIGS. 9a and 9b illustrate an example of this deployment of increasingly longer devices until the deployed length of the device is longer than the vessel section marked Lᵥₑₛₛₑₗ on FIG. 9b.

The determination of the first selection of the plurality of implantable medical devices based on a first suitability metric further includes, determining a first selection of the plurality of implantable medical devices in which the difference between the length of the implantable medical device in the deployed configuration and the target length of the target location is below a length difference threshold.

A particularly advantageous implementation of the method of using two suitability metrics in a two-step process is described as follows.

Firstly, as described above, the maximum vessel diameter within the target location is determined from the model of the vascular structure. This excludes a portion of the vessel comprising the aneurysm. The aneurysm may be identified as described above, based on a discrepancy in the radius of the vessel (for example, as compared to the centerline radius). The maximum vessel diameter is therefore the largest vessel diameter excluding the aneurysm neck or any bifurcations.

Secondly, at least one device is selected from the database, where the device model has a diameter that is greater than the maximum vessel diameter. As described above, the database comprises one or more different implantable device models. Each of these is available in a range of diameter sizes and, at each diameter size, a range of lengths. Therefore, a single device may be selected with an appropriate diameter for subsequent simulation of a range of lengths at that diameter. More preferably, a number of devices are selected that have the correct sized diameter to take forward into the subsequent simulation at various lengths. These may correspond to a number of different models from different manufacturers that have the appropriate diameter for the target vessel. They may also include a number of different diameter sizes of the same model to be taken forward for the subsequent simulations. In particular, the software may not only select the devices with the smallest diameter above the maximum diameter but optionally, all those devices within a specified diameter range. In this way, a number of diameter sizes of each model may be taken forward into the subsequent simulations. The diameter range may be varied to vary the number of devices which are selected. At the end of this step, the method has identified one or more devices, preferably a plurality of devices which may be different models having the same diameter, a single model with different diameter sizes or a number of different models with a number of different diameter sizes, all of which fulfill the required diameter range criterion.

Thirdly, for each of these plurality of devices, an initial length size is selected where the initial length size is smaller than the length of the target location. The smallest available length size may be selected as the initial length size or alternatively a length size is selected that is a percentage or absolute value shorter than the target location. The deployed configuration is then simulated for the initial length size to determine the deployed length. If the deployed length falls within a target range of the length of the target location (the “target length”) the device (i.e., the specific model, diameter size and length) is taken forward for determination of the second suitability metric. In this way, the correspondence between the deployed length and the target length can be considered a first suitability metric, where only if this meets the requirements is the device taken forward to the calculation of the second suitability metric (which may be more computationally intensive). If the deployed length does not fall within the target length range, an increased length size is then simulated (e.g., the next length size up from the initial length) to determine whether it falls within the target range. As described above, stepping up through the length sizes in this manner is computationally efficient as it only requires adding smaller additional components onto the already simulated device. The process of increasing the length size of the device and resimulating is continued until a device (of that model and diameter) is determined as meeting the first requirement (the length correspondence). This process is then repeated for each of the remainder plurality of devices (of different models and/or different diameter sizes) to determine a selection of the plurality of devices for which the second suitability metric will be determined.

In a fourth step, each of the selected plurality of devices which meet the length requirement is then simulated to determine its deployed configuration and a calculation of the second suitability metric. This may be a more computationally intensive calculation where a large number of points on the device must be calculated, to determine an accurate measure of, for example, the apposition index, landing zone index or porosity index. The second suitability metric is calculated for each of the selected devices and the best fitting device or a ranking of a plurality of best fitting devices is displayed together with a numerical value of their suitability metric and/or the constituent parameters.

In an alternative embodiment, instead of step 210, the plurality of simulated implantable medical devices are ranked based on their individual suitability metrics, as determined in step 208. The user may be provided the ranking of the devices in a form suitable for the user to interpret. Optionally, the user may be able to weigh different aspects of the suitability metric, such as the porosity index, such that the ranking of the devices promotes devices with particularly essential metrics.

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

For example, although the examples illustrated in the figures use the example of a stent to illustrate the methods, the method is equally applicable to other types of implantable device. For example, the method may equally be applied to intrasaccular devices for implantation within an aneurysm. Each of the above parameters may be calculated for an intrasaccular device. In this case, rather than a “length” of the device relative to a length of a target vessel location, other dimensions are used. Notably three dimensions defining the internal shape of the aneurysm and the corresponding dimensions of the device.

It will be appreciated that the processes described above can be implemented using a computer. Here, ‘computer’ is understood in the broad sense to refer to any collection of processing resources capable of operating on digital data. This includes traditional physical computers such as laptops, desktop computers, tablets, mobile phones, etc., and also virtual computers such as cloud-based virtual machines, servers, server clusters, and the like.

As used herein, the term “non-transitory computer-readable media” is intended to be representative of any tangible computer-based device implemented in any method or technology for short-term and long-term storage of information, such as, computer-readable instructions, data structures, program modules and sub-modules, or other data in any device. Therefore, any one or more steps of the methods described herein may be encoded as executable instructions embodied in a tangible, non-transitory, computer readable medium, including, without limitation, a storage device, and/or a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including, without limitation, volatile and non-volatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

As will be appreciated based on the foregoing specification, the above-described embodiments of the disclosure may be implemented using computer programming or engineering techniques including computer software, firmware, hardware or any combination or subset thereof, wherein the technical effect is enabling the planning of deployment of an implantable medical device, such that a clinician is able to take into account many of the important factors for deployment, such as wall apposition, porosity, obstructions of flow, and vessel geometry. Additionally, providing a computationally efficient method by which to carry out this planning of the deployment of an implantable medical device. Any such resulting program, having computer-readable code means, may be embodied or provided within one or more computer-readable media, thereby making a computer program product, i.e., an article of manufacture, according to the discussed embodiments of the disclosure. The article of manufacture containing the computer code may be made and/or used by executing the code directly from one medium, by copying the code from one medium to another medium, or by transmitting the code over a network.

1-20. (canceled)

21. A computer-implemented method for simulating a deployment of a plurality of implantable medical devices to determine a best fit implantable medical device for a target deployment location within a patient vascular structure, the method comprising:
receiving image data corresponding to the patient's vascular structure;
creating a three-dimensional model of the patient's vascular structure based on the image data;
simulating the deployed configuration of the plurality of implantable medical devices at a target location within the three-dimensional model of the patient's vascular structure;
determining a suitability metric for each simulated implantable medical device, the suitability metric providing a measure of the fit of the deployed implantable medical device within the patient's vascular structure and comprising a measure of the apposition between the implantable medical device in the deployed configuration and a wall of the vascular structure; and
outputting an indication of a best fit implantable medical device based on the suitability metric.

22. The computer-implemented method of claim 21, wherein the suitability metric comprises a measure of the correspondence between a dimension of the implantable medical device in the deployed configuration and a corresponding dimension of the target location.

23. The computer-implemented method of claim 21, wherein the measure of the apposition comprises an apposition index comprising a percentage of the surface of the implantable medical device that is within a first threshold distance from the wall of the vascular structure.

24. The computer-implemented method of claim 23, wherein the target location comprises a vessel comprising an aneurysm, and wherein the apposition index is calculated by:
determining a portion of the vessel corresponding to the aneurysm neck;
excluding the portion of the outer surface of the implantable medical device corresponding to the determined portion of the vessel;
calculating the apposition index over the remainder of the outer surface of the implantable medical device.

25. The computer-implemented method of claim 21, wherein:
the target location comprises a vessel comprising an aneurysm;
the suitability metric comprises a landing zone index; and
the landing zone index comprises a measure of the length of the landing zones of the implantable medical device, wherein the landing zones comprise longitudinal sections of the deployed implantable medical device positioned immediately distally and proximally to an aneurysm neck in the target location, and wherein the landing zone indicates a better fitting device where the landing zone length is above a threshold value.

26. The computer-implemented method of claim 21, wherein:
the suitability metric comprises a porosity index;
the porosity index comprises a measure of the porosity of the flow diverting device in the deployed configuration; and
the target location comprises a vessel comprising an aneurysm; and
wherein the porosity index is calculated by:
determining a portion of the vascular structure corresponding to an aneurysm neck;
selecting the portion of the outer surface of the deployed implantable medical device corresponding to the determined portion of the vessel;
determining the porosity of the selected portion of the outer surface of the deployed implantable medical device; and
wherein the porosity index indicates a better fitting device where the porosity is below a threshold value over the selected portion of the outer surface.

27. The computer-implemented method of claim 21, wherein the suitability metric comprises one or more of:
an obstruction value providing a measure of the degree to which the deployed implantable medical device obstructs side branches within the three-dimensional model of the vascular structure, the obstruction value indicating a better fitting device where fewer side branches are obstructed; and
a vessel geometry value providing a measure of the degree to which the deployed implantable medical device extends across one or more bends in the three-dimensional model of the vascular structure that comprise a radius of curvature below a threshold, the vessel geometry value indicating a better fitting device where fewer bends are present that are below the radius of curvature threshold.

28. The computer-implemented method of claim 21, wherein the suitability metric comprises a flow diversion index, and wherein the flow diversion index is calculated by:
simulating blood flow through the patient's vascular structure without the presence of an implantable medical device;
simulating blood flow through the patient's vascular structure including the deployed configuration of the implantable medical devices; and
determining a flow diversion index comprising a measure of the change in blood flow due to the deployed implantable medical device.

29. The computer-implemented method of claim 21, wherein the target location comprises a vessel comprising an aneurysm, and wherein the suitability metric comprises a neck protrusion value providing a measure of the distance that the deployed implantable medical device protrudes outside of a neck of the aneurysm into the vessel, the neck protrusion value indicating a better fitting device where it tends toward zero.

30. The computer-implemented method of claim 21, wherein simulating the deployed configuration of the plurality of implantable medical devices at a target location within the three-dimensional model of the patient's vascular structure comprises numerically simulating an expansion of the implantable medical device within the three-dimensional model of the patient's vascular structure.

31. The computer-implemented method of claim 21, wherein simulating the deployed configuration of the plurality of implantable medical devices at a target location within the three-dimensional model of the patient's vascular structure comprises simulating the deployed configuration of the plurality of implantable medical devices sequentially in order of unconstrained dimensions of the implantable medical device.

32. The computer-implemented method of claim 21, wherein simulating the deployed configuration of the plurality of implantable medical devices at a target location within the three-dimensional model of the patient's vascular structure comprises:
extracting a centerline from the three-dimensional model of the patient's vascular structure, wherein the centerline is a middle axis of a vessel within the patient's vascular structure; and
numerically simulating an expansion of the implantable medical device along the centerline within the three-dimensional model of the patient's vascular structure.

33. The computer-implemented method of claim 21, further comprising numerically simulating an expansion of the implantable medical device within the three-dimensional model of the patient's vascular structure based on one or more of the implantable medical device properties, geometrical constraints posed by the patient's vascular structure, and forces applied to the implantable medical device by the patient's vascular structure.

34. The computer-implemented method of claim 21, further comprising:
determining a first selection of the plurality of implantable medical devices based on a first suitability metric; and
determining a best fit device within the first selection of implantable medical devices based on a second suitability metric.

35. The computer-implemented method of claim 34, wherein determining a first selection of the plurality of implantable medical devices based on a first suitability metric comprises:
simulating the deployed configuration of the plurality of implantable medical devices and determining the difference between the length of the implantable medical device in the deployed configuration and a length of the target location; and
determining a first selection of the plurality of implantable medical devices in which the difference between the length of the implantable medical device in the deployed configuration and the length of the target location is below a length difference threshold.

36. The computer-implemented method of claim 35, wherein simulating the deployed configuration of the plurality of implantable medical devices comprises simulating the deployed configuration of the plurality of implantable medical devices sequentially in order of increasing unconstrained length of the implantable medical device.

37. The computer-implemented method of claim 21, wherein prior to simulating the deployed configuration of the plurality of implantable medical devices, the plurality of implantable medical devices are selected by:
determining a maximum vessel diameter in a target location of the three-dimensional model, excluding a portion of the target location corresponding to an aneurysm; and
accessing a database of candidate devices and selecting the plurality of implantable medical devices to be simulated as the implantable medical devices within the database having an unconstrained diameter within a target range based on the maximum vessel diameter.

38. The computer-implemented method of claim 21, further comprising determining a best fit device within a first selection of implantable medical devices based on a second suitability metric.

39. The computer-implemented method of claim 21, wherein the plurality of implantable medical devices comprise a plurality of implantable neurovascular medical devices.

40. A non-transitory computer-readable medium comprising instructions stored thereon, which when executed by a processor, causes the processor to:
receive image data corresponding to the patient's vascular structure;
create a three-dimensional model of the patient's vascular structure based on the image data;
simulate the deployed configuration of the plurality of implantable medical devices at a target location within the three-dimensional model of the patient's vascular structure;
determine a suitability metric for each simulated implantable medical device, the suitability metric providing a measure of the fit of the deployed implantable medical device within the patient's vascular structure and comprising a measure of the apposition between the implantable medical device in the deployed configuration and a wall of the vascular structure; and
output an indication of a best fit implantable medical device based on the suitability metric.
